(19) United States
(12) Patent Application Publication
(10) Pub. No.: US 2020/0046986 A1
(43) Pub. Date: Feb. 13, 2020

(54) SYSTEMS AND METHODS FOR POWERING MEDICAL DEVICES

(71) Applicants: ARVIN CHANG, Yorba Linda, CA (US); Scott Pool, Laguna Hills, CA (US); Blair Walker, Mission Viejo, CA (US)

(72) Inventors: ARVIN CHANG, Yorba Linda, CA (US); Scott Pool, Laguna Hills, CA (US); Blair Walker, Mission Viejo, CA (US)

(21) Appl. No.: 16/537,392

(22) Filed: Aug. 9, 2019

Related U.S. Application Data
(60) Provisional application No. 62/716,720, filed on Aug. 9, 2018.

(51) Int. Cl. A61N 1/378; A61B 5/00

(52) U.S. Cl. CPC A61N 1/3787; H02J 50/12; A61B 2560/0219; A61B 5/0031

(57) ABSTRACT

A wirelessly-powered medical system includes an intermediate energy transfer module configured to receive energy transferred from a main power source, a transferring magnetic resonator connected to the intermediate energy transfer module and configured to transmit a wireless power transfer signal, and a medical device configured for placement on, in, or in close proximity to a subject, the medical device requiring at least some input power to be fully operational, the medical device connected to a receiving magnetic resonator, wherein the transferring magnetic resonator is configured to exchange power wirelessly via the wireless power transfer signal with the receiving magnetic resonator.

### INCORPORATION BY REFERENCE TO ANY PRIORITY APPLICATIONS

This application claims the benefit of priority to U.S. Provisional Patent Application No. 62/716,720, filed on Aug. 9, 2018, which is herein incorporated by reference in its entirety for all purposes. Priority is claimed pursuant to 35 U.S.C. § 119.

### FIELD OF THE INVENTION

The field of the invention generally relates to charging, powering, and control systems for medical devices utilizing power.

### SUMMARY OF THE INVENTION

In one embodiment of the present disclosure, a wirelessly-powered medical system includes an intermediate energy transfer module configured to receive energy transferred from a main power source, a transferring magnetic resonator connected to the intermediate energy transfer module and configured to transmit a wireless power transfer signal, and a medical device configured for placement on, in, or in close proximity to a subject, the medical device requiring at least some input power to be fully operational, the medical device connected to a receiving magnetic resonator, wherein the transferring magnetic resonator is configured to exchange power wirelessly via the wireless power transfer signal with the receiving magnetic resonator.

In another embodiment of the present disclosure, a wirelessly-powered medical system includes an intermediate energy transfer module configured to receive energy transferred from a main power source, a transferring magnetic resonator connected to the intermediate energy transfer module and configured to transmit a wireless power transfer signal, a medical device including a skin-placeable temperature sensor configured for placement on, in, or in close proximity to a subject, the medical device requiring at least some input power to be fully operational, the medical device connected to a receiving magnetic resonator, wherein the transferring magnetic resonator is configured to exchange power wirelessly via the wireless power transfer signal with the receiving magnetic resonator, and a main power source configured to transfer energy to the intermediate energy transfer module.

In yet another embodiment of the present disclosure, a wirelessly-powered medical system includes an intermediate energy transfer module configured to receive energy transferred from a main power source, a transferring magnetic resonator connected to the intermediate energy transfer module and configured to transmit a wireless power transfer signal, a medical device including an infant monitor configured for placement on, in, or in close proximity to a subject, the medical device requiring at least some input power to be fully operational, the medical device connected to a receiving magnetic resonator, wherein the transferring magnetic resonator is configured to exchange power wirelessly via the wireless power transfer signal with the receiving magnetic resonator, and a main power source configured to transfer energy to the intermediate energy transfer module.

In still another embodiment of the present disclosure, a wirelessly-powered medical system includes an intermediate energy transfer module configured to receive energy transferred from a main power source, a transferring magnetic resonator connected to the intermediate energy transfer module and configured to transmit a wireless power transfer signal, and a medical device including a skin-placeable temperature sensor configured for placement on, in, or in close proximity to a subject, the medical device requiring at least some input power to be fully operational, the medical device connected to a receiving magnetic resonator, wherein the transferring magnetic resonator is configured to exchange power wirelessly via the wireless power transfer signal with the receiving magnetic resonator.

### BRIEF DESCRIPTION OF THE DRAWINGS

FIG. 1 is a plan view of a wirelessly-powered medical device system according to an embodiment of the present disclosure.

FIG. 2 is a front view of a mobile device of the wirelessly-powered medical device system of FIG. 1.

FIG. 3 is a side view of the mobile device of FIG. 2.

FIG. 4 is an inverter of the wirelessly-powered medical device system of FIG. 1.

FIG. 5 is a detail view of a medical device of the medical device system of FIG. 1.

FIG. 6 is a plan view of a diagnostic medical implant according to an embodiment of the present disclosure.

FIGS. 7A-7C are plan views of therapeutic medical implants according to embodiments of the present disclosure.

FIG. 8 is a perspective view of a wirelessly-powered medical device system according to an embodiment of the present disclosure.

FIG. 9 is a perspective view of a wirelessly-powered medical device system according to an embodiment of the present disclosure.

FIG. 10 is a plan view of a combination system comprising a wireless power network and a wirelessly-powered medical device system according to an embodiment of the present disclosure.

FIG. 11 is a plan view of a combination system comprising a wireless power network and a wirelessly-powered medical device system according to an embodiment of the present disclosure

FIG. 12 is a plan view of a combination system comprising a wireless power network and a wirelessly-powered medical device system according to an embodiment of the present disclosure.

### DETAILED DESCRIPTION OF THE ILLUSTRATED EMBODIMENTS

The present disclosure relates to wirelessly-powered medical device systems, including medical device systems comprising implants. Many medical devices are implanted within a patient, or are worn on or adjacent the skin of the patient. Many of these medical implants require power to operate, commonly, electrical power. Medical implants that require electrical power for operation are often comprise one or more batteries or power cells that have a finite life. At the end of the life of the battery, a surgical incision can be made to access and replace the battery, or in some cases to replace the battery and the entire implant. In some cases, rechargeable batteries have been used. However, the charging of the battery has typically required a skin incision or puncture to make a physical electrical contact between the implanted battery and an external charging unit. In some cases, batteries have been chargeable via inductive coupling by placing a coil connected to the implant subcutaneously in the patient, and placing another coil of the charging unit on the outer surface of the patient's skin, immediately adjacent the subcutaneous coil. In some cases, the charging process may be used to charge a capacitor instead of a battery. In other cases, the inductive coupling process may be used to directly power a circuit of the implant, thus avoiding the charging of a battery. The distance between the external coil and the implanted coil typically needs to be rather small, in order to allow sufficient power to be delivered to the implanted coil. In some cases, unacceptable heating occurs because of the need for high electrical currents to supply sufficient power. Heating can be damaging to the patient, and so lower currents may be used, but this may increase the charge time or the inductive powering time to an unacceptably long length.

Medical devices that are placed externally to the patient, but which are carried on or adjacent the skin of the patient, often have performance requirements that preclude wires or wired connections to the externally-placed medical device. Medical devices attached to the skin are often very low profile, and may need to be flexible. In some of these devices, adhesion between the device and the skin may need to be maintained for a long period of time. Devices of this nature that also require continual handling for connecting and disconnecting electrical wires would be disadvantageous. Some medical devices may also be used externally to a patient, but in close proximity. These devices may not necessarily by touching or immediately adjacent the skin of the patient. For example, an infant monitor or baby monitor is often configured to provide a video image of an infant and/or an audio signal in order to monitor the infant's immediate status for parents or caregivers in a separate room, or even in a separate building or location. Advanced infant monitors may even be made to acquire a continual or even continuous non-touch temperature measurement of the infant. In some embodiments, the infant monitors acquire the temperature using infrared thermometers. Though these monitors may be external, and capable of being powered through an electrical cable, it may be desired that the infant monitor be positioned very close to the infant in order to more readily achieve its purpose. An infant monitor having wires may be kicked or grabbed by the infant, potentially affecting its operation. An electrically-chargeable infant monitor that has no attached wires or jacks may be an improvement that minimizes operational problems. In all cases, a medical device that can be charged or powered wirelessly can be quite useful.

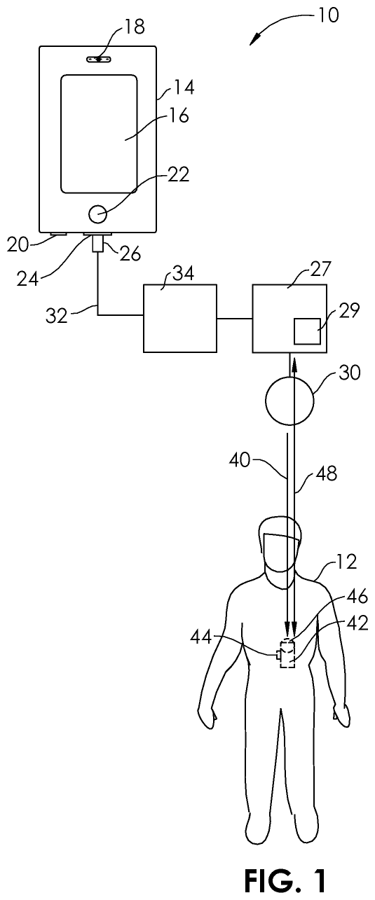

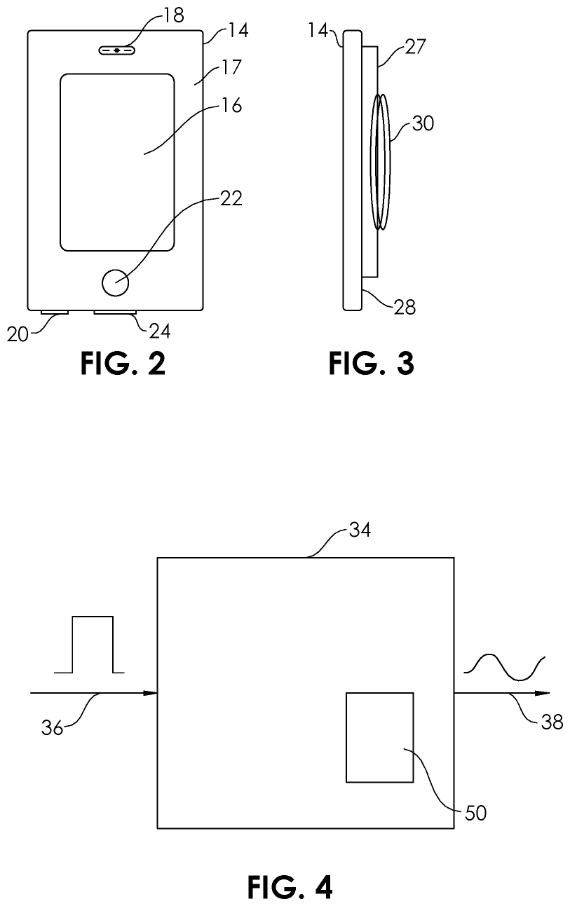

FIG. 1 illustrates a wirelessly-powered medical device system 10 for use with a patient 12. The wirelessly-powered medical device system 10 includes a mobile device 14, which may comprise a computing device, such as a mobile telephone (e.g., a smartphone), a tablet computer, a personal computer, a laptop computer, an infant monitor, or a watch or bracelet-shaped computing device. The mobile device 14 may have a touch screen 16, an audio speaker 18, a microphone 20, an on/off button 22, and a jack 24, into which a connector 26 may be attached. The touch screen 16 is carried on a front face 17 of the mobile device 14. Turning to FIGS. 2 and 3, the mobile device 14 is connected to a power transfer module 27, which is shown in FIG. 3 secured on a rear face 28 of the mobile device 14. A magnetic resonator 30 is carried on the power transfer module 27 and electrically coupled thereto. The mobile device 14 is configured to output a DC voltage signal 36 (FIG. 4) out pins in the jack 24 and through the connector 26 and a cable 32 or wire, and then into an inverter 34. The inverter 34 is configured to transform the DC voltage signal 36 to an AC voltage signal 38. The inverter 34 outputs the AC voltage signal 38 to the power transfer module 27 (FIG. 1), to be transmitted wirelessly by the magnetic resonator 30. The patient 12 has a medical device 42, shown in FIG. 1 as an implant. The medical device 42 includes a rechargeable battery 44 and a magnetic resonator 46. To charge the medical device 42, a wireless power transfer signal 40 is transmitted by the magnetic resonator 30 to the magnetic resonator 46. Returning to FIG. 4, the inverter 34 may include a circuit 50, which may comprise an oscillator circuit having a transformer which is configured to allow adjustment of the output AC voltage signal 38 in relation to the input DC voltage signal 36. The power transfer module 27 of FIG. 3 may also enclose the connector 26, the cable 32, and/or the inverter 34. The wireless power exchange between the magnetic resonator 30 and the magnetic resonator 46 may comprise highly resonant wireless power transfer (HR-WPT). Systems and methods for achieving an optimized highly resonant wireless power transfer (HR-WPT) are found in U.S. Pat. No. 9,106,203, entitled “Secure wireless energy transfer in medical applications,” issued Aug. 11, 2015, which is hereby incorporated by reference in its entirety for all purposes. The power transfer module 27 may include a processor 29 that is configured to improve or optimize the cooperation between the magnetic resonator 30 and the magnetic resonator 46. The processor 29 may comprise a microprocessor. The processor 29 can be configured to adjust a frequency of the wireless power transfer signal. The processor 29 can be configured to make adjustment related to impedance, in order to adjust the impedance matching between the magnetic resonator 30 and the magnetic resonator 46. In some embodiments, the magnetic resonator 30 is coupled to a first impedance matching network and the magnetic resonator 46 is coupled to a second impedance matching network. The magnetic resonator 30 is configured to be effective in transferring wireless power to the magnetic resonator 46 when the separation between the magnetic resonator 30 and the magnetic resonator 46 of the implanted medical device 42 is 1.5 meters or less. Thus, in most medical implant applications, the mobile device 14 can be located on or adjacent the patient 12 (externally) such that it close enough to allow wireless power transfer to an implanted medical device 42. In some embodiments, the magnetic resonator 30 is configured to be effective in transferring wireless power to the magnetic resonator 46 when the separation between the magnetic resonator 30 and the magnetic resonator 46 of the implanted medical device 42 is 1.0 meter or less. In some embodiments, the magnetic resonator 30 is configured to be effective in transferring wireless power to the magnetic resonator 46 when the separation between the magnetic resonator 30 and the magnetic resonator 46 of the implanted medical device 42 is 0.5 meter or less. As the magnetic power transfer is substantially non-radiative, a high level of safety can be provided, even through the resonators 30, 46 are close to the patient 12. In some embodiments, the mobile device 14 may be charged by plugging into a standard AC outlet via a charger. In some embodiments, the mobile device 14 may be remain plugged into a standard AC outlet during use. In other embodiments, the mobile device 14 may be charged by inductive coupling, for example, inductive coupled charging from another mobile device.

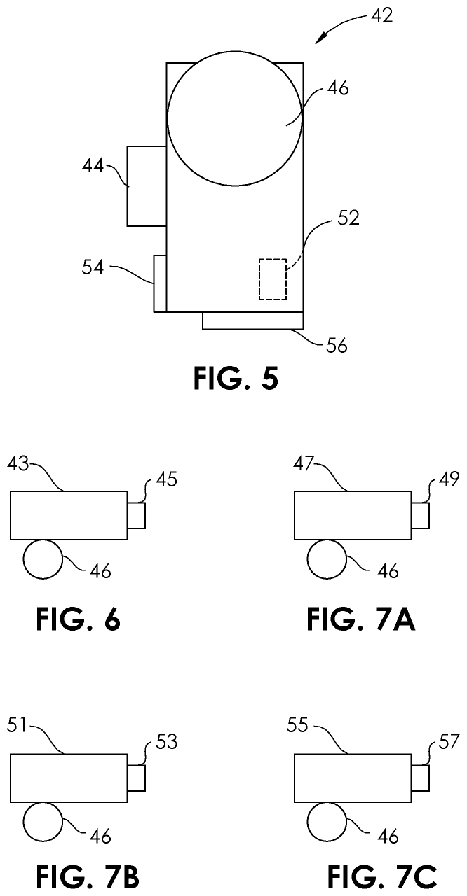

Two-way wireless data signals 48 allow the mobile device 14 and the medical device 42 to communicate with each other. Communication between the mobile device 14 and the medical device 42 may include control operations, wherein a user, such as the patient or a physician, medical personnel, friend, or family member, uses the mobile device 14 to make changes to the operation of the medical device 42. In some embodiments, the medical device 42 may have diagnostic capabilities, to measure patient parameters, such as temperature, force or moment, length between structures, angle between structures, humidity, or other biological characteristics. In some embodiments, the medical device 42 may have therapeutic capabilities, to change patient parameters, such as temperature, force or moment, length between structures, angle between structures, humidity, or other biological characteristics. The medical device 42 having therapeutic capabilities may comprise a drug pump (e.g., insulin pump), a dynamic female urinary incontinence device, a dynamic male urinary incontinence device, an adjustable cosmetic implant, a cochlear implant, a neurological stimulator, including a neurological stimulator or a gastric stimulator, an active or adjustable intra-uterine device, an active or adjustable intraocular lens, an active or adjustable intrastromal corneal ring segment. A medical device 42 that is an implant would preferably have its battery 44 (or other chargeable or powerable member) located in an area that is not within or surrounded by a flowing bloodstream. For example, active cardiovascular implants such as artificial heart valves having sensors or similarly-placed devices (immersed within significant blood flow) can be prone to inaccuracy in the coupling or impedance matching between the magnetic resonator 30 and the magnetic resonator 46, because of interference by the significant number of moving ions in the bloodstream. In addition, changes to blood flow from the biological feedback within a patient would exacerbate this inaccuracy. Standard pacemakers or implantable defibrillators (including implantable cardioverter-defibrillators), on the other hand, whose battery or powering portions are not implanted within the bloodstream, but rather subcutaneously, subfascially, or submuscularly, would indeed be amendable to the medical device 42 embodiments described herein. Turning to FIG. 5, in some embodiments, the medical device 42 may require DC voltage for certain elements of its operation, and thus AC to DC voltage conversion may be done via a rectifier 52 within the medical device 42. The medical device 42 includes a diagnostic module 54, configured for performing any of the diagnostic functions described herein. The medical device 42 further includes a therapeutic module 56 configured for performing any of the therapeutic functions described herein.

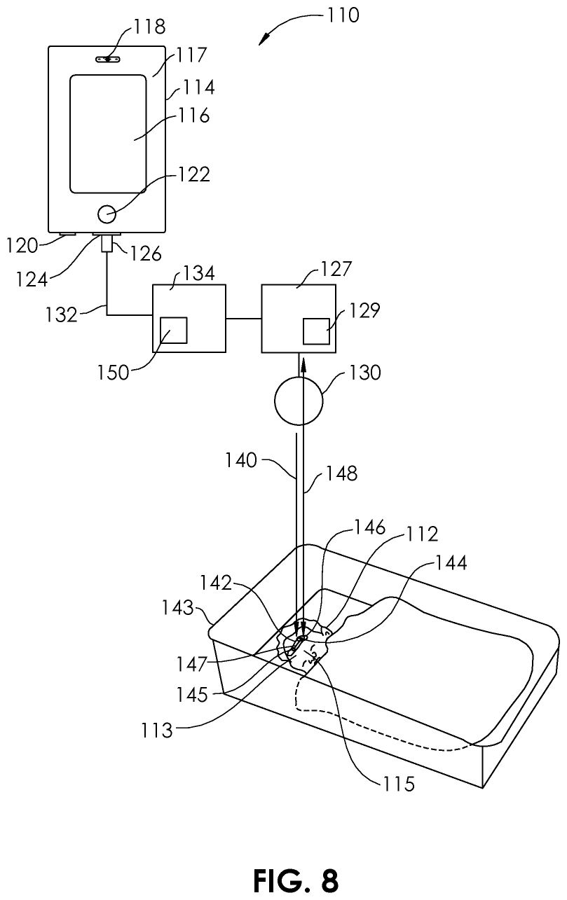

The medical device 42, in configured to be implanted within a patient, may be implanted through one or more incisions, or may be implanted by inserting through one or more natural orifices (nostril, ear canal, anus, vagina, urethra, mouth). FIG. 6 illustrates a diagnostic medical implant 43 having a sensor 45 configured for performing a measurement. The diagnostic medical implant 43 is configured to be used as the medical device 42 of the wirelessly-powered medical device system 10, and may be configured to measure patient parameters, such as temperature, force or moment, length between structures, angle between structures, humidity, or other biological characteristics. FIG. 7A illustrates a therapeutic medical implant 47 having a pump 49. The therapeutic medical implant 47 is configured to be used as the medical device 42 of the wirelessly-powered medical device system 10, and is configured to pump fluids within a patient. FIG. 7B illustrates a therapeutic medical implant 51 having an adjustable restriction 53. The therapeutic medical implant 51 is configured to be used as the medical device 42 of the wirelessly-powered medical device system 10, and is configured to open or close to form an artificial sphincter, or to adjust the size or caliber of an opening for the passage of fluids or materials. FIG. 7C illustrates a therapeutic medical implant 55 having at least one electrode 57. The therapeutic medical implant 55 is configured to be used as the medical device 42 of the wirelessly-powered medical device system 10, and is configured to apply electrical stimulation to a location within a patient. The at least one electrode 57 may comprise a single electrode or may comprise two or more electrodes. The at least one electrode 57 may be configured to be operated in a monopolar or a bipolar utility.

FIG. 8 illustrates a wirelessly-powered medical device system 110 for use with a patient 112. The patient 112 as shown in FIG. 8 is an infant within a bassinet 143, crib, bed, baby chair, or other similar object. The wirelessly-powered medical device system 110 includes a mobile device 114, which may comprise a computing device, such as a mobile telephone (e.g., a smartphone), a tablet computer, a personal computer, a laptop computer, an infant monitor, or a watch or bracelet-shaped computing device. The mobile device 114 may have a touch screen 116, an audio speaker 118, a microphone 120, an on/off button 122, and a jack 124, into which a connector 126 may be attached. The touch screen 116 is carried on a front face 117 of the mobile device 114. The mobile device 114 is connected to a power transfer module 127, which is coupled to the mobile device 114. A magnetic resonator 130 is carried on the power transfer module 127 and electrically coupled thereto. The mobile device 114 is configured to output a DC voltage signal 36 (see FIG. 4) out pins in the jack 124 and through the connector 126 and a cable 132 or wire, and then into an inverter 134. The inverter 134 is configured to transform the DC voltage signal 36 to an AC voltage signal 38 (see FIG. 4). The inverter 134 outputs the AC voltage signal 38 to the power transfer module 127 (FIG. 8), to be transmitted wirelessly by the magnetic resonator 130. The patient 112 has a medical device 142, shown in FIG. 8 as a strip secured to the skin 113 of the forehead 115. The medical device 142 includes a wirelessly powerable circuit 144 and a magnetic resonator 146. To power the medical device 142, a wireless power transfer signal 140 is transmitted by the magnetic resonator 130 to the magnetic resonator 146. The inverter 134 may include a circuit 150, which may comprise an oscillator circuit having a transformer which is configured to allow adjustment of the output AC voltage signal 38 in relation to the input DC voltage signal 36 (see FIG. 4). The power transfer module 127 may also enclose the connector 126, the cable 132, and/or the inverter 134. The wireless power exchange between the magnetic resonator 130 and the magnetic resonator 146 may comprise highly resonant wireless power transfer (HR-WPT). Systems and methods for achieving an optimized highly resonant wireless power transfer (HR-WPT) are found in U.S. Pat. No. 9,106,203, entitled “Secure wireless energy transfer in medical applications,” issued Aug. 11, 2015. The power transfer module 127 may include a processor 129 that is configured to improve or optimize the cooperation between the magnetic resonator 130 and the magnetic resonator 146. The processor 129 may comprise a microprocessor. The processor 129 can be configured to adjust a frequency of the wireless power transfer signal. The processor 129 can be configured to make adjustment related to impedance, in order to adjust the impedance matching between the magnetic resonator 130 and the magnetic resonator 146. In some embodiments, the magnetic resonator 130 is coupled to a first impedance matching network and the magnetic resonator 146 is coupled to a second impedance matching network. The magnetic resonator 130 is configured to be effective in transferring wireless power to the magnetic resonator 146 when the separation between the magnetic resonator 130 and the magnetic resonator 146 of the medical device 142 is 1.5 meters or less. Thus, it is possible to locate the magnetic resonator 130, as well as the mobile device 114 and the components therebetween, far enough from the patient 112, so that the patient 112 (e.g., infant) cannot accidentally touch or hit the magnetic resonator 130 of the other elements. In some embodiments, the magnetic resonator 130 is configured to be effective in transferring wireless power to the magnetic resonator 146 when the separation between the magnetic resonator 130 and the magnetic resonator 146 of the implanted medical device 142 is 1.0 meter or less. In some embodiments, the magnetic resonator 130 is configured to be effective in transferring wireless power to the magnetic resonator 146 when the separation between the magnetic resonator 130 and the magnetic resonator 146 of the implanted medical device 142 is 0.5 meter or less. As the magnetic power transfer is substantially non-radiative, a high level of safety can be provided, even though the resonators 130, 146 are close to the patient 112. In some embodiments, the mobile device 114 may be charged by plugging into a standard AC outlet via a charger. In some embodiments, the mobile device 114 may be remain plugged into a standard AC outlet during use. In other embodiments, the mobile device 114 may be charged by inductive coupling, for example, inductive coupled charging from another mobile device.

Two-way wireless data signals 148 allow the mobile device 114 and the medical device 142 to communicate with each other. Communication between the mobile device 114 and the medical device 142 may include control operations, wherein a user, such as the patient or a physician, medical personnel, friend, or family member, uses the mobile device 114 to make changes to the operation of the medical device 142. The patient 112, may alternatively be an adult in long-term care, hospice care, memory care, intensive care, or other modes of care. Though the word “patient” is used, the patient 112 need not be truly sick, as the medical device 142 may simply be used for monitoring or adjunctive health therapy. In some embodiments, the medical device 142 may have diagnostic capabilities, to measure patient parameters, such as temperature, force or moment, length between structures, angle between structures, humidity, or other biological characteristics. As shown in FIG. 8, the medical device 142 is adhesively-attachable strip including a temperature sensor 145. The temperature sensor 145 is electrically connected to the wirelessly-powerable circuit 144 via wiring 147. The medical device 142 may itself be a flex circuit. In some embodiments, the medical device 142 may have therapeutic capabilities, to change patient parameters, such as temperature, force or moment, length between structures, angle between structures, humidity, or other biological characteristics.

In some embodiments, the medical device 142 may require DC voltage for certain elements of its operation, and thus AC to DC voltage conversion may be done via a rectifier (as described in relation to the rectifier 52 of FIG. 5).

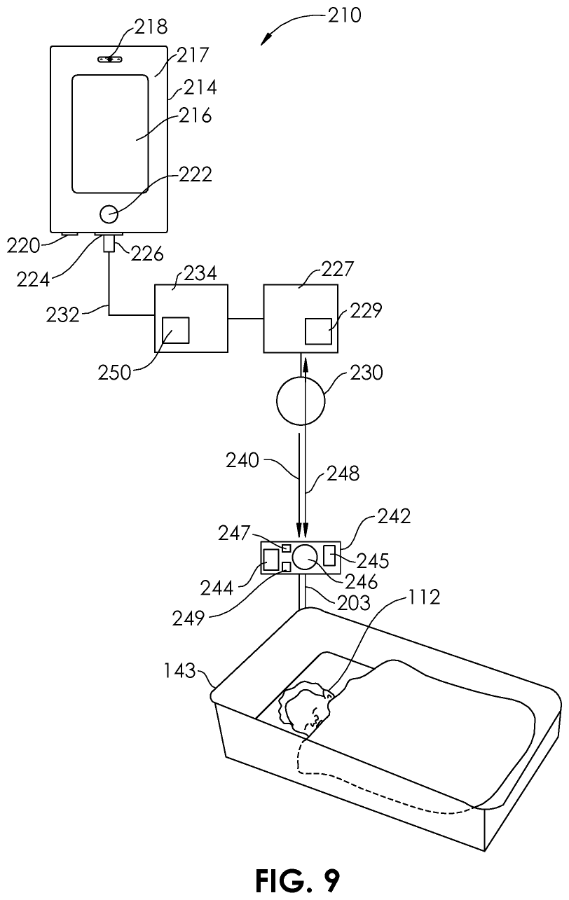

FIG. 9 illustrates a wirelessly-powered medical device system 210 for use with a patient 112. The patient 112 as shown in FIG. 9 is an infant within a bassinet 143, crib, bed, baby chair, or other similar object. The wirelessly-powered medical device system 210 includes a mobile device 214, which may comprise a computing device, such as a mobile telephone (e.g., a smartphone), a tablet computer, a personal computer, a laptop computer, or a watch or bracelet-shaped computing device. The mobile device 214 may have a touch screen 216, an audio speaker 218, a microphone 220, an on/off button 222, and a jack 224, into which a connector 226 may be attached. The touch screen 216 is carried on a front face 217 of the mobile device 214. The mobile device 214 is connected to a power transfer module 227, which is coupled to the mobile device 214. A magnetic resonator 230 is carried on the power transfer module 227 and electrically coupled thereto. The mobile device 214 is configured to output a DC voltage signal 36 (see FIG. 4) out pins in the jack 224 and through the connector 226 and a cable 232 or wire, and then into an inverter 234. The inverter 134 is configured to transform the DC voltage signal 36 to an AC voltage signal 38 (see FIG. 4). The inverter 234 outputs the AC voltage signal 38 to the power transfer module 227 (FIG. 9), to be transmitted wirelessly by the magnetic resonator 230. The patient 112 has a medical device 242, shown in FIG. 9 as an infant monitor. The infant monitor (medical device 242) is configured to be out of the reach of the patient 112 (infant). As shown in FIG. 9, the medical device 242 can be secured the bassinet 143 via a post 203. The medical device 242 includes a wirelessly powerable circuit 244 and a magnetic resonator 246. To power the medical device 242, a wireless power transfer signal 240 is transmitted by the magnetic resonator 230 to the magnetic resonator 246. The inverter 234 may include a circuit 250, which may comprise an oscillator circuit having a transformer which is configured to allow adjustment of the output AC voltage signal 38 in relation to the input DC voltage signal 36 (see FIG. 4). The power transfer module 227 may also enclose the connector 226, the cable 232, and/or the inverter 234. The wireless power exchange between the magnetic resonator 230 and the magnetic resonator 246 may comprise highly resonant wireless power transfer (HR-WPT). Systems and methods for achieving an optimized highly resonant wireless power transfer (HR-WPT) are found in U.S. Pat. No. 9,106,203, entitled “Secure wireless energy transfer in medical applications,” issued Aug. 11, 2015. The power transfer module 227 may include a processor 229 that is configured to improve or optimize the cooperation between the magnetic resonator 230 and the magnetic resonator 246. The processor 229 may comprise a microprocessor. The processor 229 can be configured to adjust a frequency of the wireless power transfer signal. The processor 229 can be configured to make adjustment related to impedance, in order to adjust the impedance matching between the magnetic resonator 230 and the magnetic resonator 246. In some embodiments, the magnetic resonator 230 is coupled to a first impedance matching network and the magnetic resonator 246 is coupled to a second impedance matching network. The magnetic resonator 230 is configured to be effective in transferring wireless power to the magnetic resonator 246 when the separation between the magnetic resonator 230 and the magnetic resonator 246 of the medical device 242 is 1.5 meters or less. Thus, it is possible to locate the magnetic resonator 230, as well as the mobile device 214 and the components therebetween, on, for example, a separate table, or on a nearby wall. The components may also be mobile within a particular area nearby. In some embodiments, the magnetic resonator 230 is configured to be effective in transferring wireless power to the magnetic resonator 246 when the separation between the magnetic resonator 230 and the magnetic resonator 246 of the implanted medical device 242 is 1.0 meter or less. In some embodiments, the magnetic resonator 230 is configured to be effective in transferring wireless power to the magnetic resonator 246 when the separation between the magnetic resonator 230 and the magnetic resonator 246 of the implanted medical device 242 is 0.5 meter or less. As the magnetic power transfer is substantially non-radiative, a high level of safety can be provided, even though the resonators 230, 246 are close to the patient 112. In some embodiments, the mobile device 214 may be charged by plugging into a standard AC outlet via a charger. In some embodiments, the mobile device 214 may be remain plugged into a standard AC outlet during use. In other embodiments, the mobile device 214 may be charged by inductive coupling, for example, inductive coupled charging from another mobile device.

Two-way wireless data signals 248 allow the mobile device 214 and the medical device 242 to communicate with each other. Communication between the mobile device 214 and the medical device 242 may include control operations, wherein a user, such as the patient or a physician, medical personnel, friend, or family member, uses the mobile device 214 to make changes to the operation of the medical device 242. The patient 112, may alternatively be an adult in long-term care, hospice care, memory care, intensive care, or other modes of care. Though the word “patient” is used, the patient 112 need not be truly sick, as the medical device 242 may simply be used for monitoring or adjunctive health therapy. In some embodiments, the medical device 242 may have diagnostic capabilities, to measure patient parameters, such as temperature, force or moment, length between structures, angle between structures, humidity, or other biological characteristics. As shown in FIG. 9, the medical device 242 may include a camera 245, such as a digital camera and may be configured to acquire video data of the patient 112. A microphone 247 may be configured to acquire audio data of the patient. A non-touch temperature sensor 249, such as an infra-red thermometer, is configured to measure a temperature of the patient 112. The camera 245, the microphone 247, and/or the non-touch temperature sensor 249 may each be wirelessly aimable (e.g., toward the patient) by control via the two-way wireless data signals 248. In some embodiments, the medical device 242 may have therapeutic capabilities, to change patient parameters, such as temperature, force or moment, length between structures, angle between structures, humidity, or other biological characteristics.

In some embodiments, the medical device 242 may require DC voltage for certain elements of its operation, and thus AC to DC voltage conversion may be done via a rectifier (as described in relation to the rectifier 52 of FIG. 5).

In some cases, the patient 112 (e.g., infant) in the embodiments of FIG. 8 or FIG. 9, may be in a first room, and the parent, parents, or other caregivers may be with the mobile device 114, 214 in a second room. In some cases, the second room may be an adjoining room to the first room. In some cases, the mobile device 114, 214 may be on the other side of a wall from the medical device 142, 242. In some cases, the magnetic resonator 130, 230 may be on the other side of a wall from the medical device 142, 242. The medical device 142, 242, thus, does not require batteries, or any wired or plugged power connection to provide continuous measurement capability. For example, a continuous temperature of the patient 112 may be viewed on the touch screen 116, 216 of the mobile device 114, 214, or may be recorded by the mobile device 114, 214, or may be audibly indicated by a loudspeaker on the mobile device 114, 116. For example, the loudspeaker may say, “ninety-eight point three degrees.” Other parameters may also be continuously measured. In other cases, the parameters may be continually measured, for example, every minute, or every two minutes, or every five minutes, or every ten minutes, or every fifteen minutes, or every 30 minutes, or every hour, or every two hours, or any other time interval.

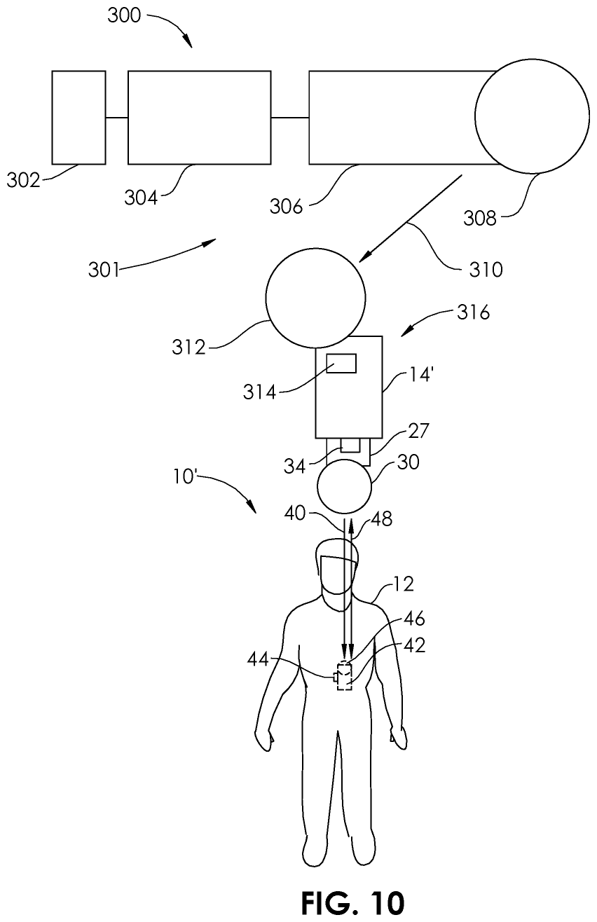

FIG. 10 illustrates a combined system 300, which includes a wirelessly-powered medical device system 10′ modified to use energy supplied by a wireless power network 301. The wireless power network 301 comprises an AC to DC converter 304 which receives an AC voltage from an AC power supply 302. The AC to DC converter 304 outputs DC voltage to an RF amplifier 306 which operates a source magnetic resonator 308. The source magnetic resonator 308 is configured to transmit a wireless power transfer signal 310 to an intermediate magnetic resonator 312 coupled to the mobile device 14′. The mobile device 14′ includes a rechargeable battery 314 configured to store electric energy delivered from the wireless power network 301 via the wireless power transfer signal 310. Other elements of the wirelessly-powered medical device system 10′ are similar to those described in relation to the wirelessly-powered medical device system 10 of FIG. 1. In some embodiments, the source magnetic resonator 308 is coupled to a first impedance matching network and the intermediate magnetic resonator 312 is coupled to a second impedance matching network. The mobile device 14′, power transfer module 27, intermediate magnetic resonator 312, and magnetic resonator 30 together comprise an intermediate device 316 configured to (1) wirelessly receive energy transferred via the wireless power signal 310 from the wireless power network 301; (2) store at least some of the received energy in the rechargeable battery 314; and (3) transmit the wireless power transfer signal 40. The intermediate device 316 is shown in FIG. 10 as a mobile device 14′, and can be configured in a large number of ways, each configured such that the intermediate device 316 can be conveniently located near the medical device 42 for wireless power transfer, but also such that the intermediate device 316 can utilize the wireless power network 301.

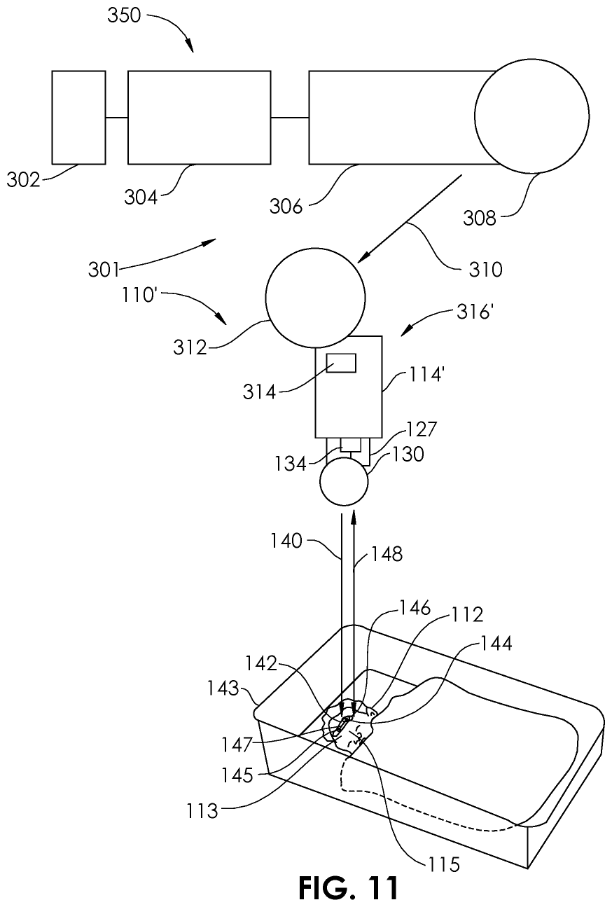

FIG. 11 illustrates a combined system 350, which includes a wirelessly-powered medical device system 110′ modified to use energy supplied by a wireless power network 301. The wireless power network 301 comprises an AC to DC converter 304 which receives an AC voltage from an AC power supply 302. The AC to DC converter 304 outputs DC voltage to an RF amplifier 306 which operates a source magnetic resonator 308. The source magnetic resonator 308 is configured to transmit a wireless power transfer signal 310 to an intermediate magnetic resonator 312 coupled to a mobile device 114′. The mobile device 114′ includes a rechargeable battery 314 configured to store electric energy delivered from the wireless power network 301 via the wireless power transfer signal 310. Other elements of the wirelessly-powered medical device system 110′ are similar to those described in relation to the wirelessly-powered medical device system 110 of FIG. 8. In some embodiments, the source magnetic resonator 308 is coupled to a first impedance matching network and the intermediate magnetic resonator 312 is coupled to a second impedance matching network. The mobile device 114′, a power transfer module 127, the intermediate magnetic resonator 312, and a magnetic resonator 130 together comprise an intermediate device 316′ configured to (1) wirelessly receive energy transferred via the wireless power signal 310 from the wireless power network 301; (2) store at least some of the received energy in the rechargeable battery 314; and (3) transmit a wireless power transfer signal 140. The intermediate device 316′ is shown in FIG. 11 as a mobile device 114′, and can be configured in a large number of ways, each configured such that the intermediate device 316′ can be conveniently located near the medical device 142 for wireless power transfer, but also such that the intermediate device 316′ can utilize the wireless power network 301.

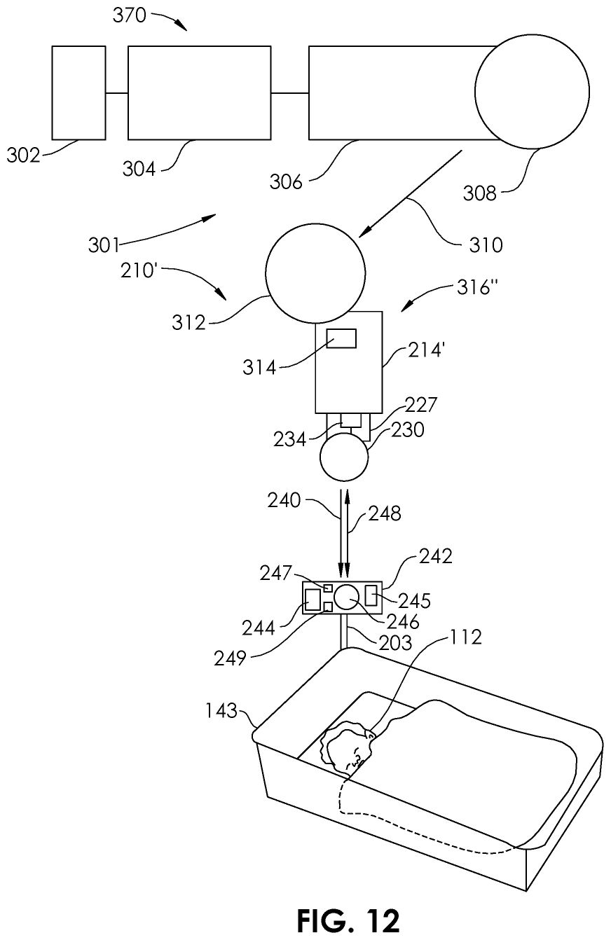

FIG. 12 illustrates a combined system 370, which includes a wirelessly-powered medical device system 210′ modified to use energy supplied by a wireless power network 301. The wireless power network 301 comprises an AC to DC converter 304 which receives an AC voltage from an AC power supply 302. The AC to DC converter 304 outputs DC voltage to an RF amplifier 306 which operates a source magnetic resonator 308. The source magnetic resonator 308 is configured to transmit a wireless power transfer signal 310 to an intermediate magnetic resonator 312 coupled to a mobile device 114′. The mobile device 114′ includes a rechargeable battery 314 configured to store electric energy delivered from the wireless power network 301 via the wireless power transfer signal 310. Other elements of the wirelessly-powered medical device system 210′ are similar to those described in relation to the wirelessly-powered medical device system 210 of FIG. 9. In some embodiments, the source magnetic resonator 308 is coupled to a first impedance matching network and the intermediate magnetic resonator 312 is coupled to a second impedance matching network. The mobile device 214′, a power transfer module 227, the intermediate magnetic resonator 312, and a magnetic resonator 230 together comprise an intermediate device 316″ configured to (1) wirelessly receive energy transferred via the wireless power signal 310 from the wireless power network 301; (2) store at least some of the received energy in the rechargeable battery 314; and (3) transmit a wireless power transfer signal 240. The intermediate device 316″ is shown in FIG. 12 as a mobile device 214′, and can be configured in a large number of ways, each configured such that the intermediate device 316″ can be conveniently located near the medical device 242 for wireless power transfer, but also such that the intermediate device 316″ can utilize the wireless power network 301.

It is contemplated that various combinations or subcombinations of the specific features and aspects of the embodiments disclosed above may be made and still fall within one or more of the embodiments. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed embodiments. Thus, it is intended that the scope of the present disclosure herein disclosed should not be limited by the particular disclosed embodiments described above. Moreover, while the present disclosure is susceptible to various modifications, and alternative forms, specific examples thereof have been shown in the drawings and are herein described in detail. It should be understood, however, that the present disclosure is not to be limited to the particular forms or methods disclosed, but to the contrary, the present disclosure is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1. A wirelessly-powered medical system, comprising:
an intermediate energy transfer module configured to receive energy transferred from a main power source;
a transferring magnetic resonator connected to the intermediate energy transfer module and configured to transmit a wireless power transfer signal; and
a medical device configured for placement on, in, or in close proximity to a subject, the medical device requiring at least some input power to be fully operational, the medical device connected to a receiving magnetic resonator, wherein the transferring magnetic resonator is configured to exchange power wirelessly via the wireless power transfer signal with the receiving magnetic resonator.

2. The system of claim 1, wherein the medical device is configured to be implanted within the subject.

3. The system of claim 2, wherein the medical device is configured to be implanted within the subject via placement through at least one surgical incision.

4. The system of claim 2, wherein the medical device is configured to be implanted within the subject via placement through one or more natural orifices.

5. The system of claim 1, wherein the medical device is configured to be secured on or adjacent the skin of the subject.

6-47. (canceled)

48. The system of claim 1, further comprising an inverter coupled to the intermediate energy transfer device.

49. The system of claim 48, wherein the inverter is configured to convert a DC voltage output from the intermediate energy transfer device to an AC signal.

50. The system of claim 48, wherein the inverter comprises an oscillator.

51. The system of claim 1, wherein the transferring magnetic resonator is carried by the intermediate energy transfer device.

52. The system of claim 51, wherein the transferring magnetic resonator has a footprint that is less than or equal to a footprint of the intermediate energy transfer device.

53. The system of claim 1, further comprising a processor carried by the intermediate energy transfer device.

54. The system of claim 53, wherein the processor is configured to make adjustments related to impedance.

55. The system of claim 54, wherein the processor is configured to transform the impedance of the transferring magnetic resonator.

56. The system of claim 1, wherein the intermediate energy transfer module comprises a control configured to wirelessly operate or adjust the medical device.

57. The system of claim 1, wherein the wireless power transfer signal is a non-radiative signal.

58. The system of claim 1, wherein the transferring magnetic resonator is configured to wirelessly exchange power with the receiving magnetic resonator when the transferring magnetic resonator is separated from the receiving magnetic resonator by a distance of 1.5 meters or less.

59. A wirelessly-powered medical system, comprising:
an intermediate energy transfer module configured to receive energy transferred from a main power source;
a transferring magnetic resonator connected to the intermediate energy transfer module and configured to transmit a wireless power transfer signal; and
a medical device comprising a skin-placeable temperature sensor configured for placement on, in, or in close proximity to a subject, the medical device requiring at least some input power to be fully operational, the medical device connected to a receiving magnetic resonator, wherein the transferring magnetic resonator is configured to exchange power wirelessly via the wireless power transfer signal with the receiving magnetic resonator.

60. The system of claim 59, wherein the skin-placeable temperature sensor is configured for placement on the skin of a subject.

61. The system of claim 59, wherein the intermediate energy transfer module comprises a control configured to wirelessly operate or adjust the medical device.

62. The system of claim 59, wherein the intermediate energy transfer module and the medical device are each configured for two-way data transmission therebetween.
